(19) United States
(12) Patent Application Publication
(10) Pub. No.: US 2025/0050125 A1
(43) Pub. Date: Feb. 13, 2025

(54) Methods and Systems for Treating Cancer Using Extremely Low Frequency Electromagnetic Fields

(71) Applicant: Balam-Quitze Ramos, Watsonville, CA (US)

(72) Inventor: Balam-Quitze Ramos, Watsonville, CA (US)

(21) Appl. No.: 18/798,746

(22) Filed: Aug. 8, 2024

Related U.S. Application Data
(60) Provisional application No. 63/531,645, filed on Aug. 9, 2023.

(51) Int. Cl. A61N 2/02; A61N 2/00

(52) U.S. Cl. CPC A61N 2/02; A61N 2/004

(57) ABSTRACT

A method of treating cancer exposes the patient to extremely low frequency electromagnetic fields (ELF-EMF) and/or extremely low frequency modulated electromagnetic fields, wherein the electromagnetic fields have frequencies ranging from 1 Hz to 300 Hz for a treatment period. The electromagnetic field frequency varies depending on the type of cancer being treated. Treatment is applied through having one or more electromagnetic coils applied to the patient or by placing the patient within an electromagnetic field generating system; and continuing the exposure for the treatment period sufficient to induce cellular changes in cancer cells of the patient, the cellular changes inhibiting the growth and proliferation of the cancer cells.

### CROSS REFERENCE TO RELATED APPLICATIONS

This utility patent application claims priority back to U.S. Provisional No. 63/531,645, filed Aug. 9, 2023, the contents of which are incorporated by reference.

### STATEMENT REGARDING FEDERALLY SPONSORED RESEARCH OR DEVELOPMENT

This invention was not federally sponsored.

### FIELD OF THE INVENTION

This disclosure is directed generally to methods of treating cancer, and more specifically to a method of treating cancer using extremely low frequency electromagnetic fields, as well as extremely low frequency acoustic and mechanical energy.

### DESCRIPTION OF THE PROBLEM SOLVED BY THIS INVENTION

The present invention provides a novel method of treating cancer using extremely low frequency electromagnetic fields (ELF-EMF), extremely low frequency acoustic and mechanical vibration, and extremely low frequency modulated electromagnetic fields. Cancer is caused by tumorous cells, which were originally normal cells that were transformed during a multi-stage process. Cancer is generally treatable at its earlier stages, but eventually progresses into malignant tumors, that become increasingly hard—and in some cases impossible—to treat. The onset of cancer is generally thought to be related to genetics combined with one or more carcinogens, which may be physical (sunlight), chemical (smoke), or biological (viruses).

Cancer is one of the leading causes of death in the world each year. The World Health Organization stated that around 10,000,000 deaths each year across the world are due to cancer. This works out to approximately one in every six deaths being attributable to cancer. Nature.com estimates that in the next 30 years, cancer will cost the world's economy over $25,000,000,000,000—twenty five trillion dollars. As a point of reference, the Burj Kalifa in UAE cost only $1.5 billion to build; thus, the world could build nearly 20,000 Burj Kalifas over the next 30 years with the money it will spend on cancer. Cancer treatments have been under development for decades, but as of today, there are no sure-fire ways to knock out cancer.

Background of the Invention. The following sections of have been taken from R. H. W. Funk et al./Progress in Histochemistry and Cytochemistry 43 (2009) 177-264. Therapeutic relevance of EMF. The use of these fields has a long history. In the first century AD, use of an electric fish was described to cure headache and gout. Later, Paracelsus (1493-1542) studied the medical use of lodestone and Sir Kenelm Digby (1603-1665) described the magnetic cure of wounds (Macklis, 1993). Modern and more serious medical applications of electromagnetic fields are to heal non-unions of bone fractures and to treat some bone-related diseases (e.g. osteoporosis, osteoarthritis), although the specific molecular mechanisms are not fully understood. The use of electromagnetic fields to stimulate osteogenesis is based on the idea of stimulating the natural endogenous streaming potentials in bone. At first, currents were applied directly via electrodes or induced by external EMF. Later, magnetic fields were produced by forcing electric currents through wire coil placed over the fracture. Periodic changes in the MF then produced the required EF in bone via Faraday induction. The most effective medical devices today use time varied (pulsed) EMF (1-100 Hz), inducing EF in the μV/cm level at the fracture site (Otter et al., 1998; Pilla, 2002). The physiological frequencies (8-30 Hz) caused by natural muscle contractions and the subsequently induced EF in bony tissue are also used for therapy. Here, these frequencies are applied as mechanical vibrations to the connective tissue and muscle (Randoll and Funk, 2004). It is important to realize that MF or EMF only induce physiological effects within certain parametric windows, i.e. ELF (8-60 Hz) and low amplitudes (≤1 G) (Gartzke and Lange, 2002). It must be pointed out that not all reports pay attention to this topic. Thus, one must have healthy skepticism.

Nevertheless, based upon multicenter, randomized and prospective clinical studies, the FDA approved pulsed EMF as safe and effective for treating nonunions and osteoporosis (Otter et al., 1998; Pilla, 2002; Chao and Inoue, 2003; Chao et al., 2004). Bone remodeling is a highly integrated process of resorption (by osteoclasts) and formation (by osteoblasts) of bone tissue that results in precisely balanced skeletal mass with renewal of the mineralized matrix. In bone diseases such as osteoporosis, the balance between bone resorption and bone formation is disturbed. Resorption outstrips formation, eventually leading to reduction in bone mineral density that enhances the risk of fracture. Regarding PEMF, most investigations have been motivated by observations of positive therapeutic results of PEMF treatment. On the other hand, PEMF frequencies and application profiles have often been ‘copied’ from the above-mentioned naturally occurring frequencies in order to give ‘healing signals’ to the body. PEMF can enhance osteoblast activity but significantly reduce osteoclast formation (Otter et al., 1998; Hartig et al., 2000; Chang et al., 2004). Thus, treatment with PEMF may shift the balance towards osteogenesis.

### The Role of Larmor Procession in Biological Targeting

In moving systems, which includes the entire organism, the Hall effect (exerted by the Lorentz force) comes into play, which is in connection to orbital electrons and electron spins (at very high flux densities: nuclear spins) and magnetic force.

Because electrons are in motion in an orbit and have spin, the torque exerted produces a change in angular momentum perpendicular to that angular momentum, causing the magnetic moment to precess around the direction of the magnetic field rather than settle down in the direction of the magnetic field. This is called Larmor precession. A bound ionic oscillator (e.g., ion bound to a protein) in a static magnetic field will precess at the Larmor frequency in the plane perpendicular to the applied field. This motion will persist in superposition with thermal forces, until thermal forces eventually separate the oscillator from a binding site. Interestingly, the high sensitivity to (alternating) EMF of only 10-100 nT, possibly is only given, if the EMF is overlaid to a static magnetic field at most in the order of the geomagnetism (≤100 μT) (see Pazur et al., 2006).

Sinusoidal EMF wave (with vector components). Middle: principle of a molecular ratchet which is able to direct the random ‘Brownian’ molecular movement if a triggering vector is present. Right: model of ion clouds ($\mathrm{Ca}\hat{ }{2+}$ ions) moving along a ratchetlike molecule (e.g., actin) and thus, leading to a polarization of charges (modified after Gartzke and Lange, 2002).

### Signal Transduction

In general, there are excellent reviews about the various effects of MF on cell biology which show a plethora of all possible effects (Adey, 1992a; Barnes and Greenebaum, 2007; Markov, 2007).

In the short history of EMF coupling to biological systems (over the last 30 years, see Pilla, 2003) the first explanations focused on ions near ion channels of the cell membrane. Here, researchers recognized that the greatest problem for EMF sensing is the random motion of ions by the thermal stochastic (Brownian) molecular movement in all 3 directions (Boltzmann ‘boundary’ discussed above). A force exerted by an EMF field should modify these 3 D vectors until finally an ion channel is triggered by this effect.

However, ions in the body are not in a free vacuum, thus it is not trivial to apply the ‘solid state physics’ approach to these problems. On has to keep in mind the ‘bound water’ situation (see Section 3.4.2.3) and the ligation of ions to the ubiquitously existing proteins or other macromolecules (including also glycoproteins and lipids).

The cell membrane is often considered to be the main target for MF signals and most results point to an MF effect on the rate of ion or ligand binding to, e.g., a receptor site acting as a modulator of signaling cascades often involving calcium/calmodulin-dependent processes, cAMP, and growth factors (see Markov, 2007). In most cases the magnetic effect is ascribed to Ca2+ ions. The biochemical reactivity of ions bound in the molecular clefts of macromolecules may be affected by SMF via changes in the spatial orientation of movement or by changing Larmor precession frequencies. For fields in the mT range, the bound lifetime must be sufficiently longer than 1 ms (Muehsam and Pilla, 1996).

For biological systems, the Larmor precession model postulates that precessional motion at the Larmor frequency of charged particles bound inside a binding site or molecular cleft can be modulated by applied MF to affect binding kinetics. This coherence modulates the fluctuations of the dielectric constant at the binding site and by this modulates the binding situation. A threshold in the 0.1-1 μT range is predicted by this model in the presence of thermal noise, with bound times of 0.5-1 s (see Pilla, 2003). The charm of this system is that no substantial input of energy or angular momentum into the system is required (Edmonds, 1993). Coherence is brought into thermal fluctuation and thus, Larmor precession may be a transduction mechanism for weak magnetic field effects on the physicochemical properties of aqueous solutions, which in turn may modulate biological responses (Edmonds, 1993; Muehsam and Pilla, 1996; Pilla et al., 1999). Furthermore, Larmor precession allows the system to be a highly sensitive detector of the MF environment in the presence of thermal noise (Pilla et al., 1997). Many studies of the Markov-Pilla group show that Ca2+-Calmodulin-dependent myosin light chain kinase and protein kinase C-dependent processes speed up to twofold (Shuvalova et al., 1991; Markov et al., 1992, 1993, 1994; Pilla and Markov, 1994; Markov and Pilla, 1994a, b, 1996, 1997; Engstrom et al., 2002; Liboff et al., 2003; see Pilla, 2007). Also, the rate of Ca2+ binding to Calmodulin was increased twofold in a 2 G SMF (Markov and Pilla, 1997; Liboff et al., 2003).

Larmor precession of water molecules may also be a generalized transduction mechanism for weak MF effects on all aqueous interactions by altering the physicochemical properties of aqueous solutions, which in turn may modulate biological responses (see Pilla, 2003 and below).

Left: sinusoidal EMF wave (with vector components). Middle: principle of a molecular ratchet which is able to direct the random ‘Brownian’ molecular movement if a triggering vector is present. Right: model of ion clouds ($\mathrm{Ca}\hat{ }{2+}$ ions) moving along a ratchetlike molecule (e.g., actin) and thus, leading to a polarization of charges (modified after Gartzke and Lange, 2002).

### Cell Lines Tested to Date and Rapid Remission

### Cell Lines Tested to Date and Rapid Remission

### Cell Lines Tested

B16F10 Melanoma cell line
Confidence: 95+%
P-Value: P<0.05
Time to Remission: 48 hours
MCF-7 Aggressive breast cancer adenocarcinoma cell lines
Confidence: 95+%
P-value: P<0.05
Time to Remission: 48 hours
MB-231 Breast Cancer Cell Line
Confidence: 95+%
P-value: P<0.05
Time to Remission: 48 hours
143B Osteosarcoma cell line
Confidence: 99.9+%
P-value: P<0.001
U-87 MG Aggressive human glioblastoma
Confidence: 99.9+%
P-value: P<0.001
PC12 Rat Adrenocarcinoma
Confidence: 99.9+%
P-value: P<0.001
Time to Remission: 48 hours
THP-1 Monocytic Leukemia
Confidence: 99.9+%
P-value: P<0.001
Time to Remission: 48 hours
MDA-MB-231 Breast Cancer Cell Line
Confidence: 95%
P-value: P<0.05
Time to Remission: 12 hours
Hepg2 Human Liver Cancer Cells
Confidence: 95+%
P-value: P<0.05
Time to Remission: 72 hours
A549 Human Nonsmall-Cell Lung Cancer Cell Lines
Confidence: 95+%
P-value: P<0.05
Time to Remission: 72 hours

Extremely low frequency electromagnetic fields (ELF-EMF) in the frequency range of around 1 Hz to 300 Hz can induce biological effects in cells. Some studies have shown that ELF-EMF exposure can inhibit cancer cell growth and proliferation by interfering with cell cycle progression, inducing apoptosis, and modulating gene and protein expression. However, there is no known technology that covers the use of ELF-EMF and ELF-modulated EMF of any frequency within the extremely low frequency range for cancer treatment.

### SUMMARY OF THE INVENTION

The present invention provides methods and systems for treating cancer by applying ELF-EMF or ELF-modulated EMF of any frequency within the extremely low frequency range to a patient, thereby inducing cellular changes that inhibit cancer cell growth and proliferation through various mechanisms of action. This method exposes the patient to extremely low frequency electromagnetic fields (ELF-EMF) and/or extremely low frequency modulated electromagnetic fields, wherein the electromagnetic fields have frequencies ranging from 1 Hz to 300 Hz for a treatment period. The electromagnetic field frequency varies depending on the type of cancer being treated. Treatment is applied through having one or more electromagnetic coils applied to the patient or by placing the patient within an electromagnetic field generating system; and continuing the exposure for the treatment period sufficient to induce cellular changes in cancer cells of the patient, the cellular changes inhibiting the growth and proliferation of the cancer cells.

### A PREFERRED EMBODIMENT OF THE INVENTION

In one embodiment, a method of treating cancer in a patient includes a first step of exposing a patient to extremely low frequency electromagnetic fields (ELF-EMF)/extremely low frequency modulated electromagnetic fields, wherein the electromagnetic fields have frequencies ranging from 1 Hz to 300 Hz for a treatment period; wherein the exposure is performed using any of various techniques for generating ELF-EMF and applying the energy to the patient in a local manner or by placing the patient within an ELF-EMF field; and a second step of repeating the exposure for the treatment period sufficient to induce cellular changes in cancer cells of said patient, the cellular changes inhibiting the growth and proliferation of the cancer cells.

Under this program, the patient is exposed to extremely low frequency electromagnetic fields (ELF-EMF)/extremely low frequency modulated electromagnetic fields. In either case, the electromagnetic field may have a simple or complex waveform containing the desired ELF spectrum. The spectrum/frequency used may be selected based on the cancer cell type.

In another preferred embodiment, the method for treating cancer calls for a first step of placing one or more electromagnetic coils, or other means of generating an EM field, configured to generate extremely low frequency electromagnetic fields (ELF-EMF) or extremely low frequency modulated electromagnetic fields with frequencies ranging from 1 Hz to 300 Hz near a patient. The method than requires a second step of providing a control system configured to activate the system to expose a cancer patient to the electromagnetic fields for a treatment period sufficient to induce cellular changes in cancer cells of the patient. This claim extends to the use of mechanical or acoustic signals at frequencies between 1 Hz and 300 Hz.

Under this second embodiment, the frequency is selected based on the cancer cell type.

Under a third embodiment, the system comprises a method for the adjunct treatment of cancer, such as an enhancer of chemotherapeutic agents or as an adjunct treatment to surgery or radiation therapy, where the ELF-EMF has demonstrated the ability to enhance the uptake of chemotherapeutic agents through various mechanisms.

There has thus been outlined, rather broadly, the more important features of the invention in order that the detailed description thereof may be better understood, and in order that the present contribution to the art may be better appreciated. There are additional features of the invention that will be described hereinafter, and which will form the subject matter of the claims appended hereto. The features listed herein, and other features, aspects and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

Various modifications to the embodiments may be apparent to one of skill in the art upon reading this disclosure. For example, persons of ordinary skill in the relevant arts will recognize that the various features described for the different embodiments can be suitably combined, un-combined, and re-combined with other features, alone, or in different combinations. Likewise, the various features described above should all be regarded as example embodiments, rather than limitations to the scope or spirit of the disclosure.

Persons of ordinary skill in the relevant arts will recognize that various embodiments can comprise fewer features than illustrated in any individual embodiment described above. The embodiments described herein are not meant to be an exhaustive presentation of the ways in which the various features may be combined. Accordingly, the embodiments are not mutually exclusive combinations of features; rather, the claims can comprise a combination of different individual features selected from different individual embodiments. as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Unless indicated otherwise, references to ‘embodiment(s)’, ‘disclosure’, ‘present disclosure’, ‘embodiment(s) of the disclosure’, ‘disclosed embodiment(s)’, and the like contained herein refer to the specification (text, including the claims, and figures) of this patent application that are not admitted prior art.

Various modifications to the embodiments may be apparent to one of skill in the art upon reading this disclosure. For example, persons of ordinary skill in the relevant arts will recognize that the various features described for the different embodiments can be suitably combined, un-combined, and re-combined with other features, alone, or in different combinations. Likewise, the various features described above should all be regarded as example embodiments, rather than limitations to the scope or spirit of the disclosure.

Persons of ordinary skill in the relevant arts will recognize that various embodiments can comprise fewer features than illustrated in any individual embodiment described above. The embodiments described herein are not meant to be an exhaustive presentation of the ways in which the various features may be combined. Accordingly, the embodiments are not mutually exclusive combinations of features; rather, the claims can comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Unless indicated otherwise, references to ‘embodiment(s)’, ‘disclosure’, ‘present disclosure’, ‘embodiment(s) of the disclosure’, ‘disclosed embodiment(s)’, and the like contained herein refer to the specification (text, including the claims, and figures) of this patent application that are not admitted prior art.

For purposes of interpreting the claims, it is expressly intended that the provisions of 35 U.S.C. 112(f) are not to be invoked unless the specific terms ‘means for’ or ‘step for’ are recited in the respective claim.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

1. A method of treating cancer in a patient, the method comprising the steps of, first, allowing the exposure of a patient to an electromagnetic field; and, second, continuing the exposure for the treatment period sufficient to induce cellular changes in cancer cells of said patient, the cellular changes inhibiting the growth and proliferation of the cancer cells.

2. The method of claim 1, where the electromagnetic field is an extremely low frequency electromagnetic fields (ELF-EMF) wherein the electromagnetic fields have frequencies ranging from 1 Hz to 300 Hz for a treatment period.

3. The method of claim 2, wherein the exposure is performed using one or more electromagnetic coils applied to the patient.

4. The method of claim 2, wherein the exposure is performed by placing the patient within an electromagnetic field generating system.

5. The method of claim 1, where the electromagnetic field is an extremely low frequency modulated electromagnetic fields, wherein the electromagnetic fields have frequencies ranging from 1 Hz to 300 Hz for a treatment period.

6. The method of claim 5, wherein the exposure is performed using one or more electromagnetic coils applied to the patient.

7. The method of claim 5, wherein the exposure is performed by placing the patient within an electromagnetic field generating system.

8. The method of claim 1, wherein the patient is exposed to extremely low frequency electromagnetic fields (ELF-EMF).

9. The method of claim 1, wherein the patient is exposed to extremely low frequency modulated electromagnetic fields.

10. The method of claim 1 wherein the electromagnetic field frequency is selected based on the cancer cell type.

11. A system for treating cancer comprising: a first step of placing one or more electromagnetic coils, or other means of generating an EM field, configured to generate extremely low frequency electromagnetic fields (ELF-EMF) or extremely low frequency modulated electromagnetic fields with frequencies ranging from 1 Hz to 300 Hz near a patient, and a second step of providing a control system configured to activate the system to expose a cancer patient to the electromagnetic fields for a treatment period sufficient to induce cellular changes in cancer cells of the patient. This claim extends to the use of mechanical or acoustic signals at frequencies between 1 Hz and 300 Hz.

12. The system of claim 5, wherein the frequency is selected based on the cancer cell type.

13. The system of claim 12, where the cancer cell type is selected from the group comprising melanoma, breast cancer, osterosarcoma, monocytic leukemia, liver cancer, and lung cancer.

14. A method for the adjunct treatment of cancer, such as an enhancer of chemotherapeutic agents or as an adjunct treatment to surgery or radiation therapy. These fields have demonstrated the ability to enhance the uptake of chemotherapeutic agents.
